(19) United States
(12) Patent Application Publication
(10) Pub. No.: US 2025/0364102 A1
(43) Pub. Date: Nov. 27, 2025

(54) MEDICATION ENVIRONMENTAL INTERACTIVE DEVICE (EID)

(71) Applicant: SANO Healthcare Consultants, LLC, Ontario, CA (US)

(72) Inventors: Edwin N. Kendrick, III, Ontario, CA (US); Christopher Lange, Arvada, CO (US); Norman Smith, II, Evans, GA (US)

(21) Appl. No.: 18/872,764

(22) PCT Filed: Jun. 7, 2023
(86) PCT No.: PCT/US2023/024714

§ 371 Date: Dec. 6, 2024

Related U.S. Application Data
(60) Provisional application No. 63/350,361, filed on Jun. 8, 2022.

(51) Int. Cl. G16H 20/13; A61J 1/03; A61J 1/14; A61J 1/16; G16H 10/60; G16H 70/40

(52) U.S. Cl. CPC G16H 20/13; A61J 1/03; A61J 1/1412; A61J 1/16; G16H 10/60; G16H 70/40; A61J 2200/30; A61J 2200/74

(57) ABSTRACT

A medication environmental interactive device (EID) is provided. The EID includes: a container having a container space for receiving a medication bottle; a multifunctional cap for attaching to the medication bottle; a weight sensor; a power supply; and programmed circuitry. The container has an interaction surface configured to receive and suspend the multi-functional cap and attached medication bottle within the container space. The weight sensor is disposed between the interaction surface and the multi-functional cap and is configured to measure the weight of the multifunctional cap, the medication bottle, and contents of the medication bottle. The programmed circuitry is programmed with instructions to receive and store information from the weight sensor.

### BACKGROUND

Smart prescription medication containers/caps of the past are equipped to determine usage by a user and/or to determine an amount of medicine contained within the container and/or that has been taken by a user. However, these containers/caps and usage thereof have been found unsatisfactory.

Smart containers require a user transfer of prescription medication from a pharmacy bottle to the container. Smart caps are configured to attach to the pharmacy bottle. In either event, sensors of the smart containers/caps are employed to detect specific actions involving the container/cap. For example, in some prior art embodiments sensors detect when a cap is removed from a container. These systems assume that this removal equates to the user taking their expected dosage of the medicine. However, this assumption is often wrong and results in poor medicine usage determinations. A user may open the container for reasons other than to dispense medication, including when the user opens the cap to check how much medicine is remaining and when the user inadvertently removes the cap.

These various sensor configurations of the art are ineffective in accurately monitoring the medicine remaining in the container and thus the medicine usage by the user. When this information is used to determine compliance by a particular user, the data is often incomplete and leads to unreliable information and unintended results.

In addition to poor medication compliance, limited patient literacy is a barrier to effective medical diagnosis and treatment. Health literacy plays a significant role in determining how well patients interpret medication labels and take their medications safely and appropriately. To assist in health literacy and adherence, the Centers for Disease Control and Prevention (CDC) Health Literacy Council recommended health communicators reduce jargon and improve a medication user's risk of hospital readmissions, hospitalizations, health complications, and death from lack of medication adherence and/or persistence.

Additionally, internet-based and health information technology (IT) in general, have the potential to create patient-centered individualized treatment tailored specifically to the end user's needs. Use of user-friendly and engaging interactive digital platforms present considerable opportunities for effectively engaging diverse populations of people with varied health literacy levels.

### SUMMARY OF INVENTION

The present invention solves the problems of the art and is directed to a medication management system, collectively comprised of a medication EID system, provisioned network communication system, and a server system. Additionally, the medication environmental interactive device (EID) may function without web-based communications through its collective interactions with external environments and internal environments as part of a self-contained medication management system until network and server communications are required for software and programming updates, wherein the medication EID contains a medication enclosure for receiving medications within a medication bottle, connects to a multifunctional cap for dispensing and preserving medications, and a SMART medication container for personalized medication management through integrative taxonomy classification systems, compliance and behavioral modification systems, power and electronics systems, and methods of forming and operating the same. The medication EID provides for accurate sensing and measurement of an amount of medicine therein contained, and safer management of medications.

In a first embodiment, the present invention provides an EID which includes: a container having a container space for receiving a medication bottle; a multifunctional cap for attaching to the medication bottle; a weight sensor; a power supply; and programmed circuitry. The container has an interaction surface configured to receive and suspend the multi-functional cap and attached medication bottle within the container space. The weight sensor is disposed between the interaction surface and the multi-functional cap and is configured to measure the weight of the multifunctional cap, the medication bottle, and contents of the medication bottle. The programmed circuitry is programmed with instructions to receive and store information from the weight sensor.

In another embodiment, the present invention provides for a medication management system including an EID, provisioned network communication systems, and a server network to manage health-related and medication-related conditions through a synchronized health portal medication management system, as described herein. The medication bottle comprises medication and is attached to the multifunctional cap. The multifunctional cap is attached to the container. The medication bottle is suspended within the container space. The weight sensor is disposed between the interaction surface and the multi-functional cap.

In further embodiments, the present invention comprises methods of using and instructions for using and/or forming the system and/or EID described herein. In yet additional embodiments, the present invention provides programmed circuitry for use with the EID and systems described herein.

### BRIEF DESCRIPTION OF DRAWINGS:

FIG. 1 shows a system embodiment of the present invention.

FIG. 2 shows another system embodiment of the present invention.

FIG. 3 shows another system embodiment of the present invention.

FIGS. 4a to 4g show different views of a medication dispenser in accordance with an embodiment of the present invention.

FIGS. 5a to 5c show different views of a multifunctional medication bottle cap dispenser in accordance with an embodiment of the present invention.

FIG. 6 shows a medication bottle having an inner ridge for interaction with a multipurpose cap.

FIG. 7 shows an exploded view of a medication dispenser in accordance with an embodiment of the present invention.

### SPECIFICATION

The present invention solves the problems of the art and is directed to a medication environmental interactive device (EID) that is functional through its interactions with external environments and internal environments as a singular and/or part of a comprehensive medication management system, wherein it contains a medication EID and/or functions with provisioned network communication systems and cloud servers housing a health portal with electronic applications, which collectively manage health and medication-related conditions as a medication management system. Additionally, the medication EID consists of a medication enclosure for receiving medications within a medication container and/or medication bottle, connects to a multifunctional cap for dispensing and preserving medications, and a SMART medication container for personalized medication management through integrative taxonomy classification systems, compliance and behavioral modification systems, power and electronics systems, and methods of forming and operating the same. The medication EID provides for accurate sensing and measurement of an amount of medicine therein contained, and safer management of medications.

In preferred embodiments, the present invention provides an enclosure for a SMART container for medication management. The SMART container includes a power supply, programmed circuitry, including a processor, a weight sensor, accelerometer, barcode scanner, capacitive touch display, and a vented container. The SMART container has vents, which maintains the suspended medication bottle at room temperature within the SMART container. This unique approach strategically supports reuse of the SMART container with any medication bottle. This approach avoids the commonly unsafe technique of users placing a different medication within a used medication bottle for the specific purpose of reusing sensors and/or load cells to assist them in remembering when and/or if a medication was taken. In one embodiment, the SMART container may receive different medication containers through use of a multifunctional cap that is suspended within the hollow walls of the SMART container. The SMART container houses a strain gauge system which measures medication through the suspended multifunctional cap engaging the interaction surface of the strain gauge system, while holding a medication container and its medication contents in a central vented chamber of the SMART container. The weight sensor is disposed between the interaction surface and the multifunctional cap, the system is configured to measure the weight of the prescription medication bottle and/or contents (e.g. a prescription medication, medication supplement) within the bottle, as well as the multifunctional suspended cap atop the strain gauge. The programmed circuitry and the processor located within the SMART container are programmed with algorithm instructions to receive and store information from the weight sensor, as well as interact with the user for medication management. In another embodiment, the present invention provides a method and system of programmed circuitry capable of receiving, storing, and initiating health-related (e.g. cardiovascular, orthopedic, endocrine, cancer, maternal-fetal medicine) and pharmacy-related (e.g. medication class, medication name, indications for use, systemic effects, side effects, interactions, safe use instructions) interactions to improve health literacy, medication adherence, and promote healthy behavioral modifications. Specifically, benefits are promoted through integrative use of a (a) taxonomy classification system which auto-populates a user interface; (b) medication adherence monitoring/dosing compliance and behavioral modification systems; (c) social engagement of care that assists providers and supportive care teams (family, providers, pharmacists, government, etc.) through a HIPAA-compliant network for receiving, sharing, and storing health information; (d) machine learning, artificial intelligence, neural networks, natural language processing, and other applicable advance data science methodologies to assess and eliminate harmful bias; and (d) a power and electrical system which promotes interactions with the SMART container and multifunctional cap to improve care access and delivery. As a result, use of the medication EID and remaining medication management systems (e.g. provisioned network communication systems; servers containing databases, algorithms, classification systems, electronic applications) support use with liquids, solids (e.g. tablets, capsules, powders), free-medications, gases, low-cost medications, and high-risk medications, in-home, remotely, and particularly in underrepresented and disabled populations, who remain at higher risk for medicine-related complications. Collectively, general preferred embodiments pertain to medication EID systems and methods for improving medication management through integration of a taxonomy classification system, compliance monitoring and behavioral modification system, and power and electrical systems, provisioned network communications, and servers.

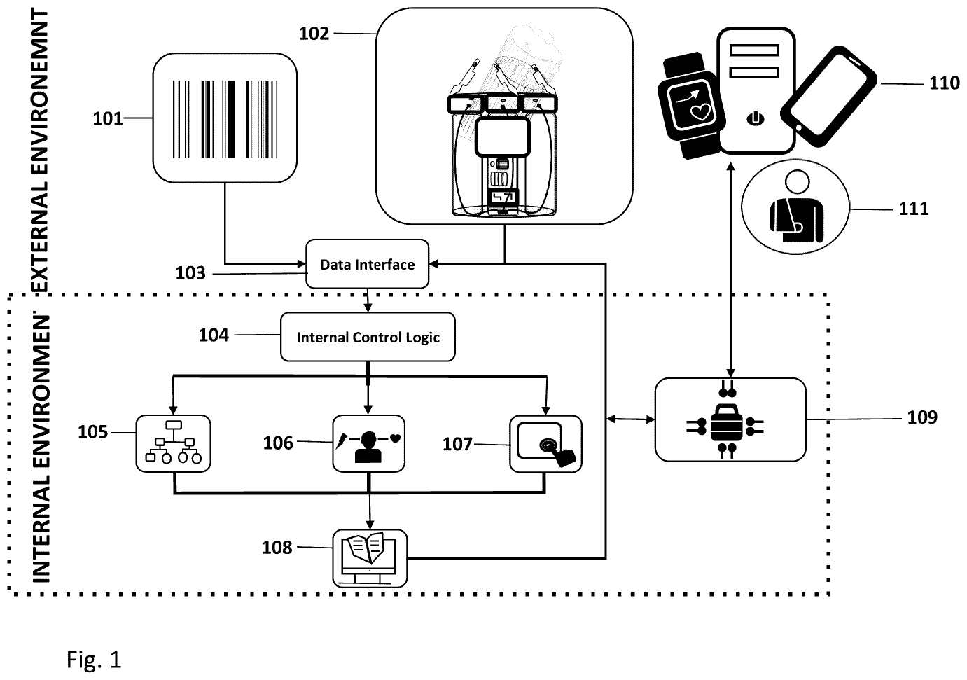

The present invention is described further in the following non-limiting exemplary embodiments and corresponding figures. In one embodiment, and as shown in FIGS. 1, 4a, 4b, and 6, the medication EID unit 102 may be activated through a barcode scanner 101 and/or the power and electrical systems 107 through an accelerometer 409, strain gauge 410, and/or algorithm control logic 108. As shown in FIG. 1, integration of a taxonomy classification system 105, compliance monitoring and behavioral modification system 106, and power and electrical systems 107 are used to improve confidence and adherence through single-hand use in persons (e.g persons with arm/hand disabilities), improving health literacy 105 and medication compliance with behavioral modification support tools (i.e. encouraging knowledge of right medication, right dose; right time, side-effects, benefits, etc.) 106, and improving patient engagement with the medication EID 103 and wireless technology tools 110.

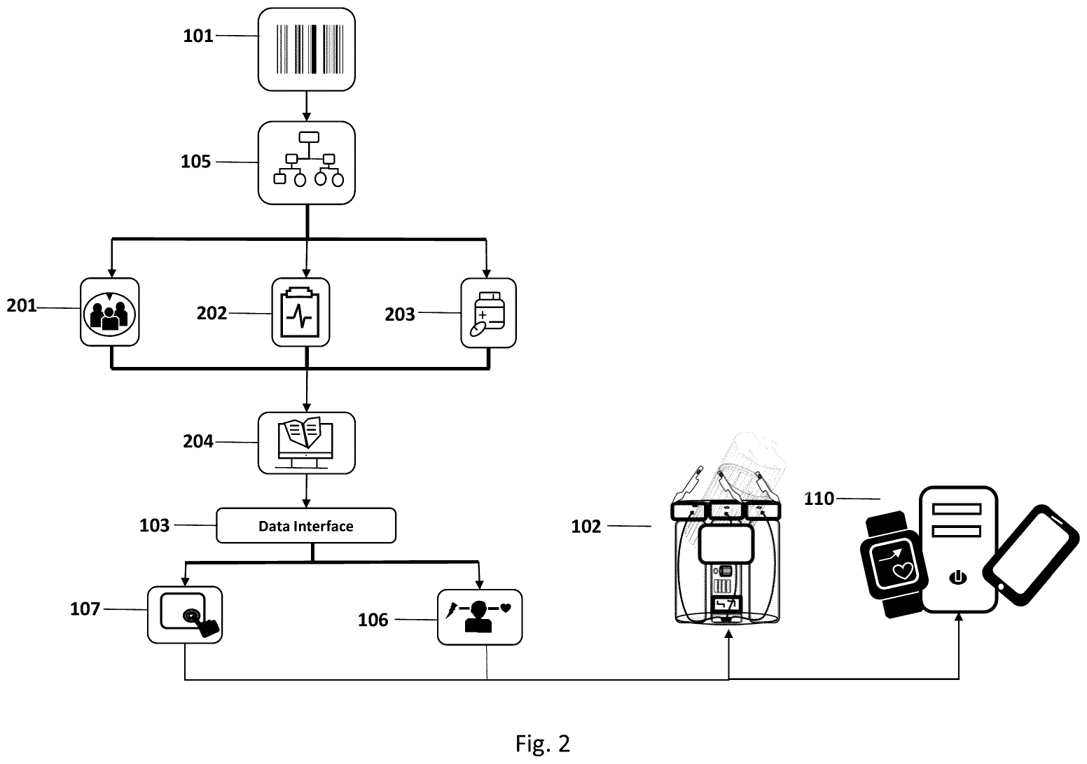

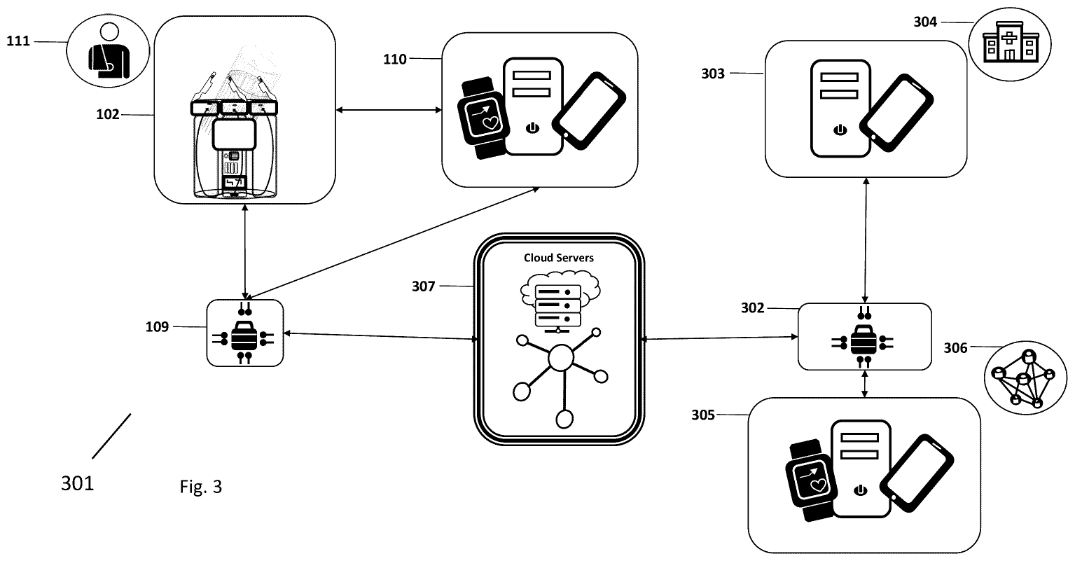

Functionally, data obtained from use of the barcode scanner 101 and/or personal action/inaction in the external environment with the medication EID 102 (e.g. taking a medication 603, moving the SMART container 401, touching the display 403, not interacting with the EID 102) will activate the internal logic system 104. The internal logic system will instruct one or more of the three integrated medication EID systems (e.g., taxonomy classification system 105, compliance monitoring and behavioral modification system 106, power and electrical systems 107) to perform an action, based upon the external environment action and/or inaction. Based upon the data received by 1 or more of the internal environment integrative systems, data will be dually stored on the microprocessor 405 while activating an algorithm control logic 108. The algorithm control logic 108 configures a medication EID 102 response that autopopulates the SMART container display 403 through the data interface 103. Hence, the algorithm control logic 108 provides alerts, and/or provides messaging to assist in improving medication adherence, medication persistence, health literacy, and reducing health complications and hospital readmissions through a continuous feedback loop. Additionally, as seen in FIG. 1 and FIG. 2, the algorithm control logic 108 operates subsets of health literacy and data science algorithm control logic 204. To assist in continuity of care and access to care, the stored activity data 108 within the microcontroller 405 may be displayed on pcu computers and wireless communication devices of the end-user 110, healthcare providers 303, and members of the end-user's approved social network 305, as shown in FIG. 3.

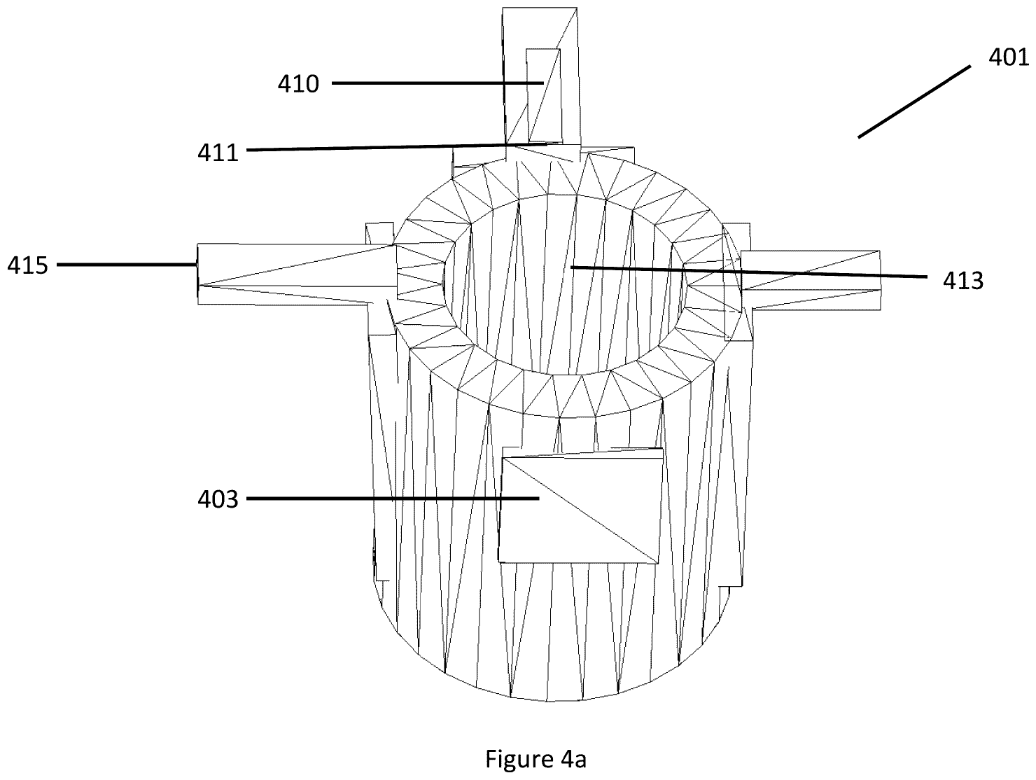

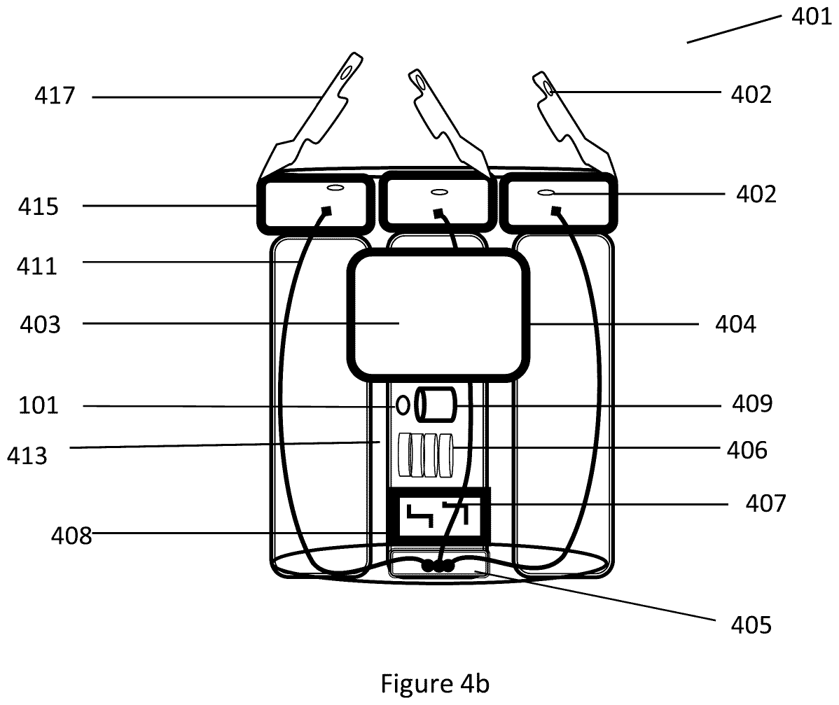

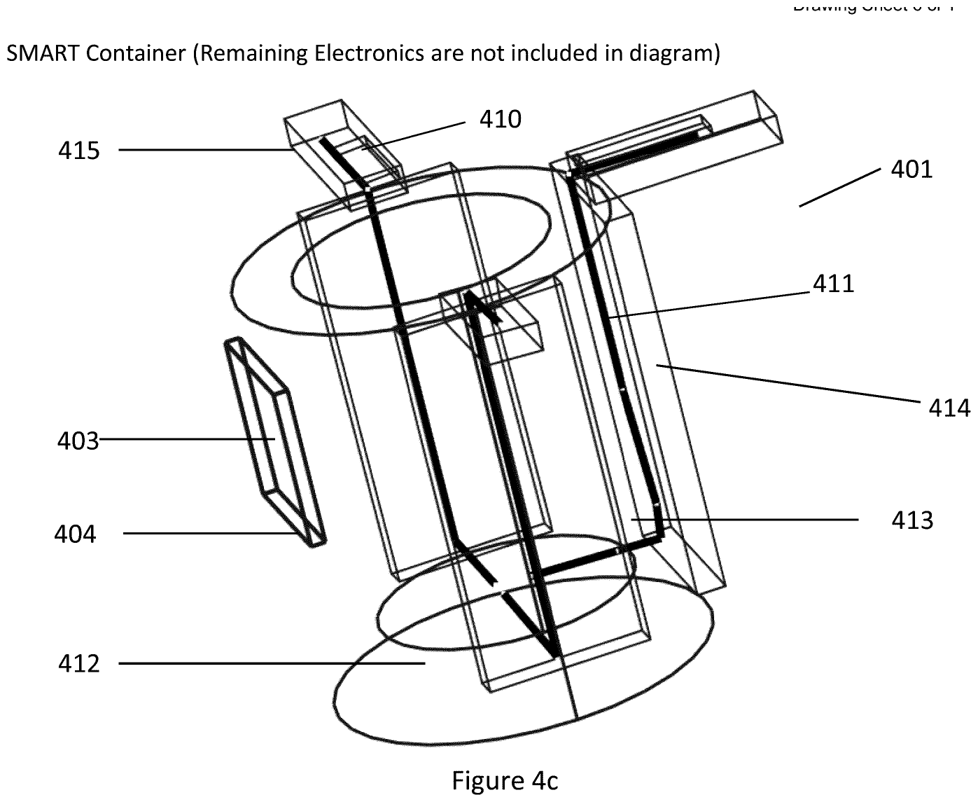

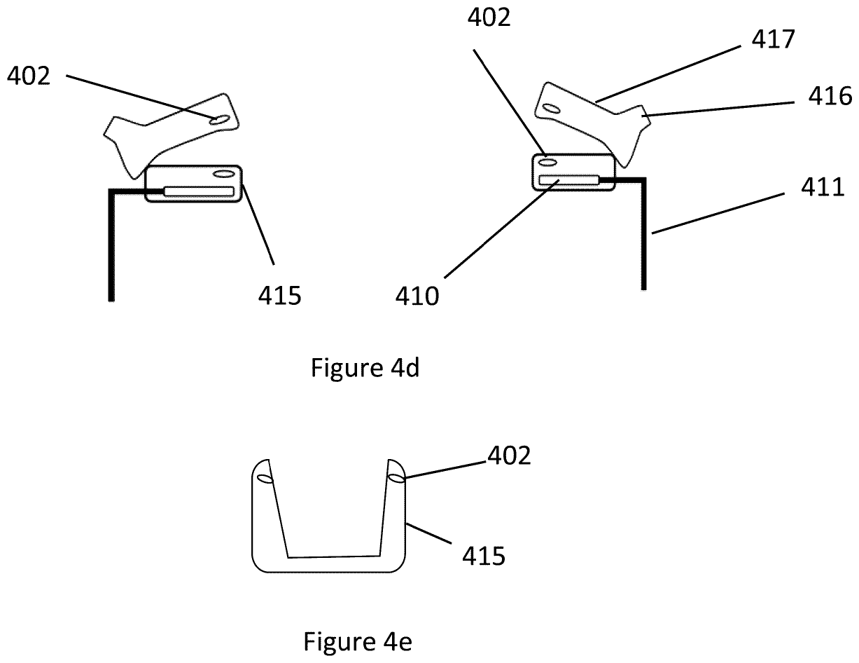

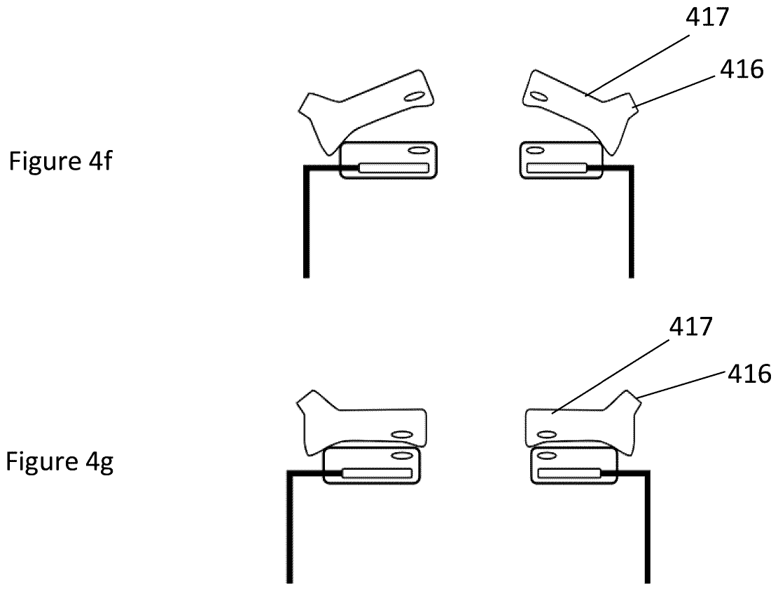

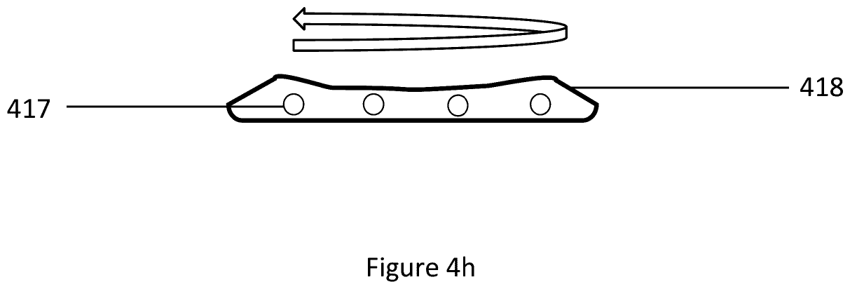

As shown in FIG. 2, in another embodiment, the taxonomy classification system (205) is designed to convert medical jargon of medical diagnoses 202, medication name 203, indications for medication 203, how the medication works 203, how the medication affects the body 203, and medication side effects 203 into plain language terms that are configured within a person-centered algorithm control logic, which overcomes common health literacy barriers by expanding preferred health literacy language choices which are tailored to an individual through a culturally sensitive classification system (205), and which adapts to a person based on artificial intelligence, machine learning, and advanced data science analytics 204. Additionally, the power and electrical systems 207 support health literacy through converting and/or providing information to the user through audio, visual, touchscreen, and by emitting a specified color (e.g. blue, green, yellow, etc.) and spectrum (e.g. darker, lighter) of light, such as red, blue, green (RGB) color chart to specify a specific class and type of medication on the medication EID display 403 and/or surrounding the display 404 as seen in FIG. 4. Specifically, for a medication with more than one clinical use, a multicolored light pattern will be displayed, with most of the light emitted from the medication EID display 403, as shown in FIG. 4, being based on the specific class of prescribed medication for a specific medical condition class (e.g., cardiovascular, endocrine, orthopedic). The emitting of light on the medication EID display 403 includes but is not limited to light emitting diode (LED) technology. The internal environment of the medication EID 202 contains health literacy and data science algorithm control logic 204 that is adaptable through use of data that provides culturally-sensitive, plain language, and behavioral modification based upon historic medication EID use 206, demographics data 201, medical condition data 202, and medication data 203, and serves as a subset logic of the algorithm control logic.

In another embodiment, to assist in continuity of care and access to care, stored activity and health data 108, as seen in FIG. 1, may additionally be shared, as seen in FIG. 3, through medication EID software, network, and server systems 301. The medication EID, the network, and the server systems 301 support person-centered control, through a network-provisioned communication system 302 that manages healthcare providers 304 (e.g., hospitals, clinical researchers, scientists, governmental agencies, social services agencies, pharmacies, nurses, physicians, etc.) and social network members 306. Specifically, the medication EID system, network system, and server system 301 function as a medication management system, facilitating use of a health platform managed by cloud servers 307, which ensure proper database configuration and updating, compliance monitoring, behavioral modification, user-controlled access, electronic data interchange for electronic health records integration, network messaging, and advanced data science methods, and updates are incorporated within the network an update an interdependent medication EID 102, the provisionally-shared networks 302 309, and server systems 307.

More specifically, the health portal system housed throughout the medication EID, networking, and server systems 301 provides end-user controls, access to health and medication alerts, evaluation of health literacy and behavioral modification data, network messaging, compliance monitoring for medication adherence, measured medication EID system benefits, and access to specific shared electronic health records when granted access by the medication EID user 311. Thus, the provisioned-network communication system 302, limits access of health providers 304 and social network members 306 to the medication EID user 311 health-related information. Medication EID software, network, and server systems 301 facilitate use of electronic applications (e.g., web based, cloud based, mobile based) that are accessible on the medication EID, pcu computers and/or personal wireless technology devices (e.g., desk-tops, lap-top computers, tablets, cell phones, Smart watches, Smart glasses, etc.) of medication EID users 310, approved healthcare providers 303, and approved social network members 305. As shown in FIG. 3, personal engagement is increased directly through the end-users' interaction with the medication EID 102 and/or their personal communication devices 310, allowing medication EID users 311 to express medication concerns and alerts directly to providers 304, caregivers 306, clinical researchers 304, family 306, and social networks 306. Additionally, when the medication EID is in proximity of a provision-network communication system of the EID user 309 and/or approved healthcare providers and social network members 302, the user's stored data 108 may be transferred to cloud servers 307 and/or relayed to the wired or wireless communication transmitter/receiver devices (e.g. communication antenna for Bluetooth, wifi).

In a further embodiment, a method of ordering medication refills from the medication EID 102 and/or through personal wireless communication devices 310 may occur, when in proximity of provisioned network communication systems of medication EID users 309. In another embodiment, user health and medication-related emergency alerts may be sent through the medication EID's capacitive touch display screen 403, as shown in FIG. 4, and/or through the medication EID user's personal wireless devices 310, where it may be retrieved by healthcare providers 304 and/or approved social network members 306.

As shown in FIG. 3, the medication EID 102 is a self-contained and functioning unit that may reside in diverse external environments of users 311, including in homes and apartments, medical facilities, and during travel/vacations.

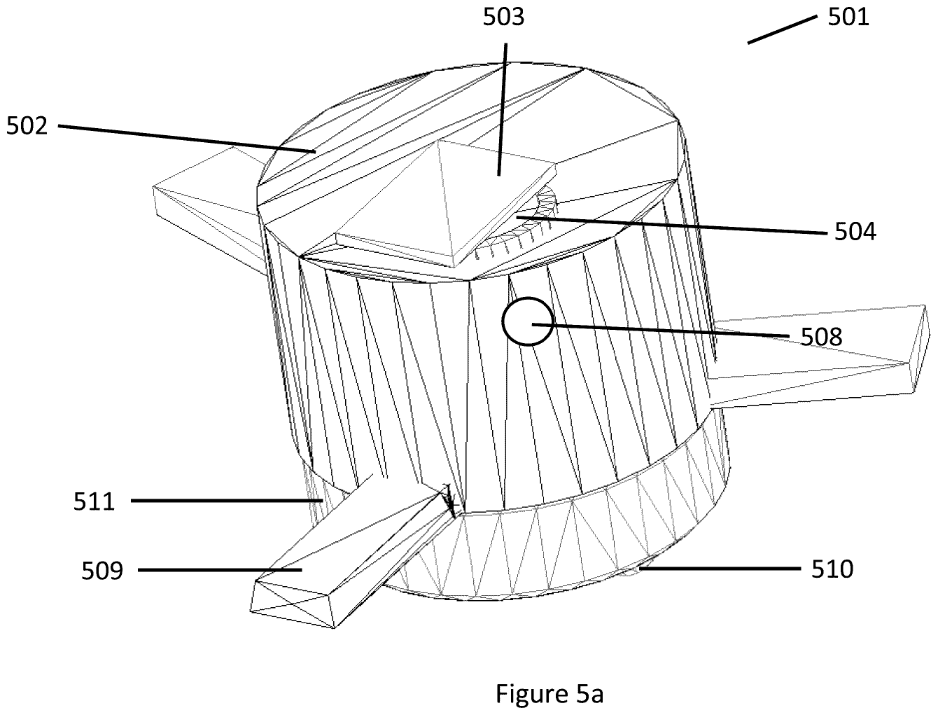

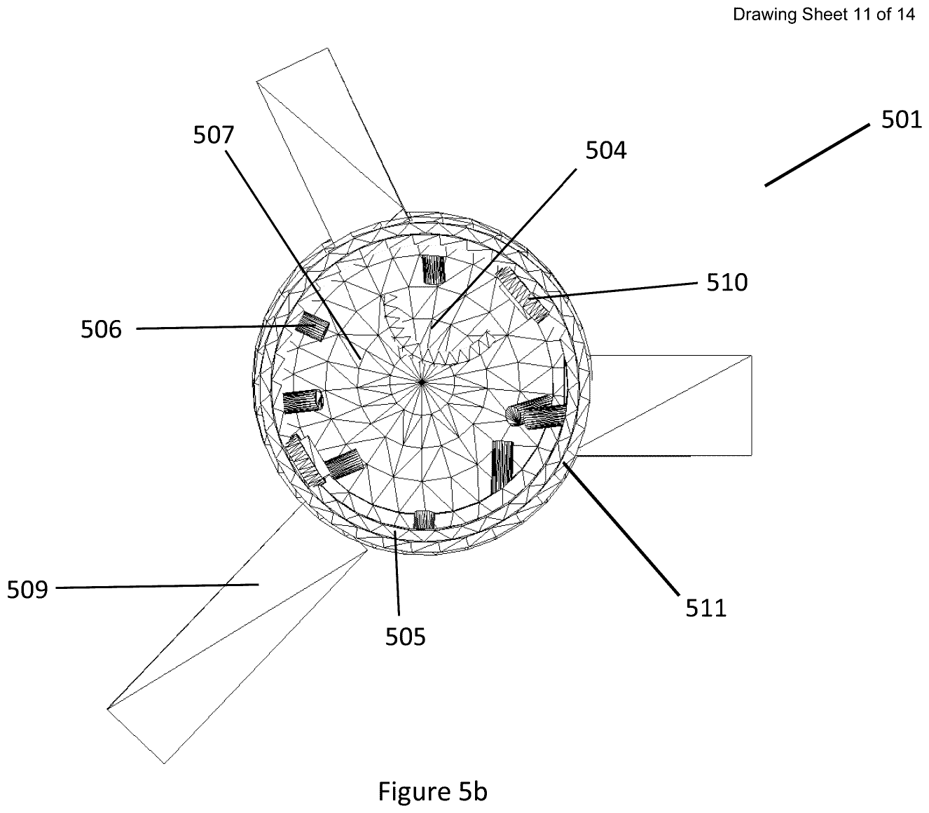

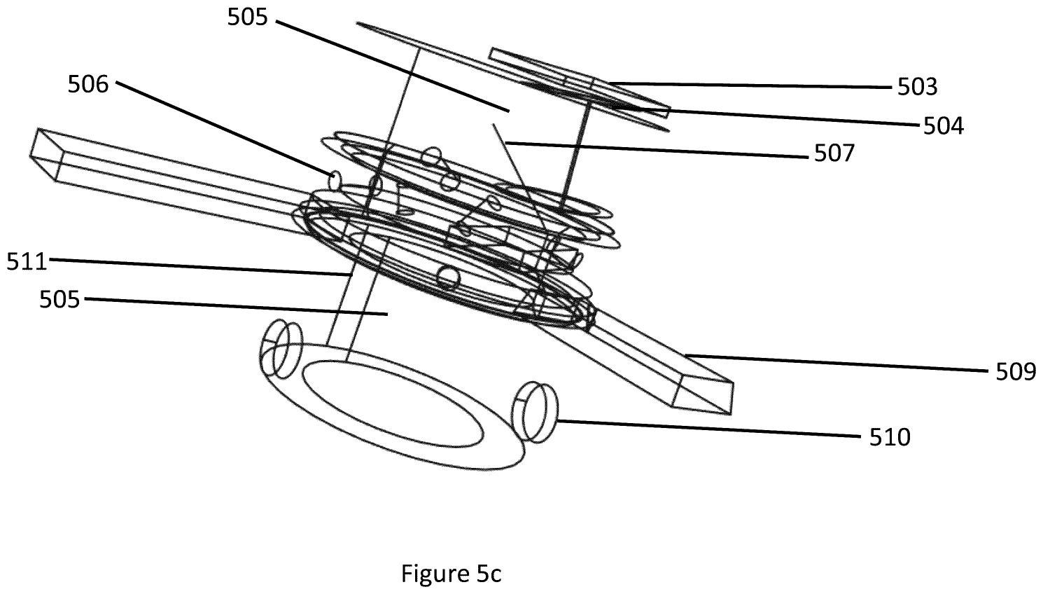

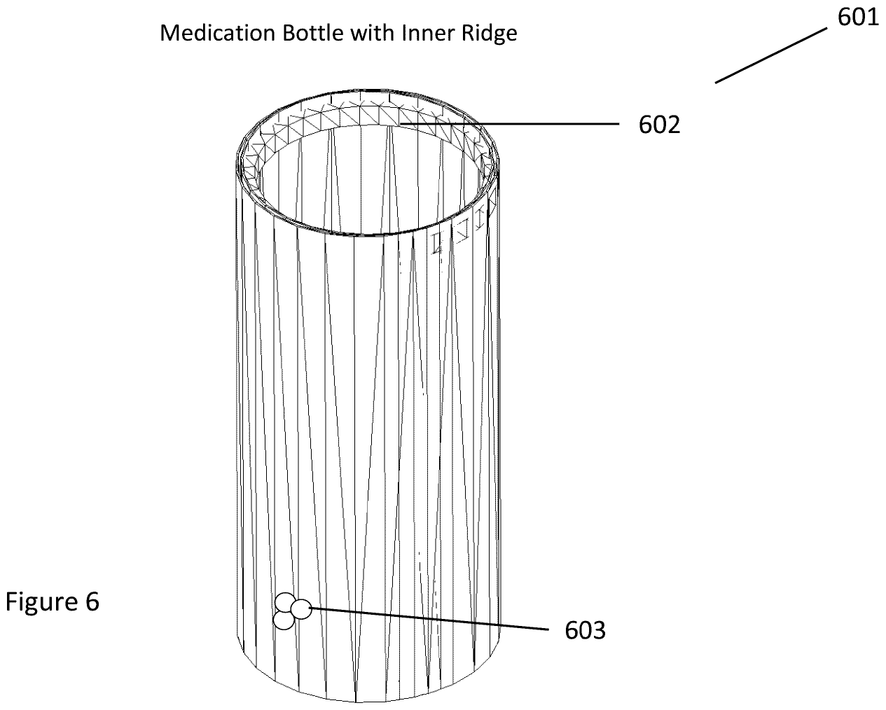

As shown in FIG. 6, in accordance with one embodiment, the medication EID is configured to receive a portable drug containment unit (medication bottle 601) which has: (a) a medication container 601 with a horizontal internal ridge 602 for securing the multifunctional cap 501, described below with respect to FIGS. 5a to 5c. The medication bottle 601 holds a prescription and/or non-prescription medication 603 and fastens to a lateral projection extending from the multifunctional cap button 510. The lid is preferably a multifunctional cap 501. As shown in FIGS. 5a to 5c, the multifunctional cap is attached to the medication bottle 601, and is additionally designed for (a) single-hand use engagement from the medication bottle 601 (when centrally pressing the multifunctional cap 501 onto the medication bottle 601 to snap fit on the underside of the inner ridge of the medication bottle 602) and disengagement of the medication bottle 601 (when pressing underneath and in an upward manner against a horizontal tab of the multifunctional cap 509 to disengage the multifunctional cap button 510 away from the inner ridge of the medication bottle 602), (b) reducing pill spills through internal medication obstructors extending from the internal walls of the funnel-shaped interior chamber of the multifunctional cap, (c) embedding food-safe silicone or similar product for dehumidification and/or as a desiccant of the medication bottle, and (d) for one hand dispensing of solid medications one at a time.

Additionally, as shown in FIGS. 4a to 4c, integration of a taxonomy classification system, compliance monitoring and behavioral modification system, and power and electrical systems, through use of a SMART Container, supports improved personal engagement and compliance.

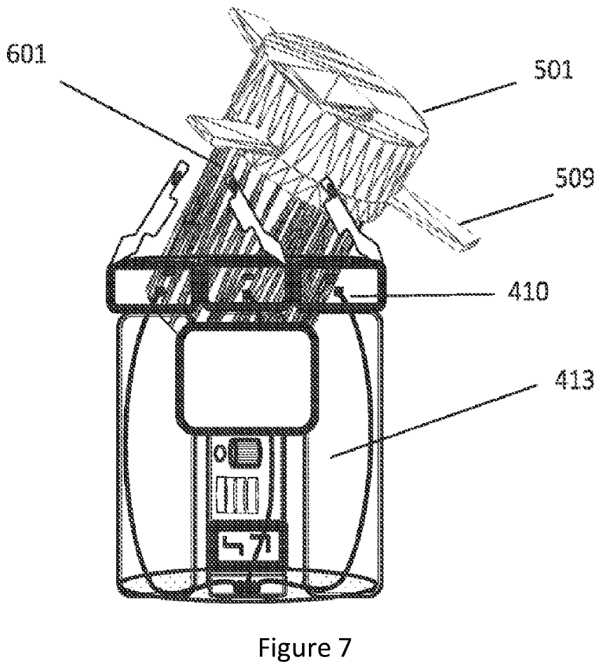

In other embodiments, as shown in FIGS. 4a to 4g, the present invention provides a SMART container 401 with a vented central container 413 and multiple hollow walls and hollow enclosures for receiving and/or securing mechanical and power/electrical systems, such as the strain gauge 410, the prescription medication bottle 601, as shown in FIG. 6, a multifunctional cap 501, as shown in FIGS. 5a to 5c, and remaining mechanical and electrical components detailed above. The SMART container has power and electrical systems that include a processor 405, power supply 406 a weight sensor (e.g. strain gauge) 410, a multifunctional cap 501 as shown in FIG. 5, and a vented central compartment 413 of the SMART container 401, which allows circulation of air from the external environment through the vents, to maintain the medication container at room temperature. Specifically, the SMART container 401 has hollow channels within wall spaces of the container for supporting passage of conducting wires 411, a display screen 403 to be housed, and remaining power and electrical systems to be maintained and enclosed within the walls, which will be accessible for repairs. The container 401 has a hollow contact surface 415 (container contact surface) which borders the vented container space 413, wherein the hollow contact surface is configured to receive the horizontal tabs of the multifunctional cap 509 atop a strain gauge 410, while the multifunctional cap 501 simultaneously suspends the attached medication container 601 within the central chamber of the vented SMART container 413, as partially shown in FIG. 7.

A weight sensor 410 is disposed between the hollow strain gauge contact surface 415 and the multifunctional cap 501, shown in FIG. 5a, and is configured to calculate the weight of the contents within the prescription medication bottle 603, shown in FIG. 6, while using an algorithm that configures pill weight while incorporating the weight of the multifunctional cap 501, the attached medication bottle 601, and the medication contents (e.g. a prescription medication) within the medication bottle 603. The programmed circuitry and the processor 405 are communicatively coupled with and programmed to receive, send, and store information from the weight sensor.

The container is not particularly limited, but will preferably be sized and shaped to accommodate different prescription bottle shapes, such as circular, rectangular, and L-shaped medication containers (e.g. pill bottles, injectables, inhalers).

Referring to all embodiments, the contents, components and/or configuration of the programmed circuitry and/or power and electrical systems is not particularly limited. In preferred embodiments, the programmed circuity will comprise electronic circuitry and/or devices such as any or all of a display, speaker, microphone, U/I interface, wired connection ports, wired or wireless communication transmitter/receiver (e.g. communication antenna for Bluetooth or other wireless communication protocol to provide notifications to social network member, medication EID users, and/or healthcare providers, etc.), accelerometer, dehumidifier, a power source, such as a battery, a processor, storage, memory and/or other circuitry and/or software or a module programmed with instructions for performing required functionality of the device as described herein.

In preferred embodiments the device further comprises a scanning device 101, such as the feature shown in FIG. 1 (e.g. linear, laser, 2D area bar code scanner), with circuity programmed to auto populate in an algorithmic derived manner and/or instruct the medication EID user through information prompting and/or through interactions with the medication EID, regarding medical condition, patient information, and/or prescription medication administration and/or similar related topics. In some embodiments, such data transmission may be accomplished through a customized application running on a smartphone, smartwatch, tablet, computer, or other future technologies (e.g. smart glasses).

While not limited, in preferred embodiments the weight sensor comprises a strain gauge 410. The type and components of the strain gauge are not particularly limited. However, preferably the strain gauge is formed from a strain gauge metal and/or a metal alloy or is formed from graphene nanosheet (GNS) product, including but not limited to carbon matter, such as a GNS synthesized using a water-assisted liquid phase exfoliation (GNS-LPE).

Referring again to FIGS. 4a to 4h, the hollow strain gauge contact surface 415 is preferably used in conjunction with one or more moveable top covers 417 attached to the hollow strain gauge contact surface 415, to engage (secure) and disengage the multifunctional cap 501, shown in FIGS. 4d-4g, between them. The magnets 402 located within the side walls 4e of the hollow strain gauge contact surface 415 and the moveable top cover 417 are in proximity of each other and disengage when the mobile top cover 417 is depressed over its vertically pointed ridge 416. The hollow strain gauge contact surface is shown in front view in FIG. 4c, while movement of the top cover(s) 417, with respect to hollow strain gauge contact surface(s) 415, are shown in FIGS. 4d, 4f and 4g.

In preferred embodiments, a horizontal tab 509 extends from the multifunctional cap, where it will fit within and/or otherwise engage the hollow strain gauge contact surface 415 of the SMART container shown in FIG. 4, atop at least 1 or more strain gauges 410, which may or may not have conduction wires 411 attached to the strain gauges 410. In other embodiments, same or similar mechanisms are employed to cover the horizontal tabs 509 of the multifunctional cap 501 shown in FIG. 5a, which includes but is not limited to use of a horizontally-rotating clasp 418, shown in FIG. 4h, with protruding externally-facing tabs 417, which fastens itself (i.e., engages) and unfastens itself (i.e, disengages) when the rotating clasp is fully rotated and is less than fully rotated upon itself. These embodiments ensure the medication bottle 601, shown in FIG. 6, and multifunctional cap 501, shown in FIG. 5a, may be securely engaged within the SMART container 401, as shown in FIG. 4a, without interfering with the weighing mechanism of the SMART container.

When the multifunctional cap 501, shown in FIG. 5a, is attached to the medication bottle 601, shown in FIG. 6, and is placed into the SMART Container 401, shown in FIG. 4a, in an engaged configuration, the tabs 509 of the multifunctional cap 501 are enclosed, secured, and/or suspended on the strain gauges within the hollow contact surface 415 of the SMART Container 401, while weight of the multifunctional cap 501, the attached medication bottle 601, and contents of the medication bottle 603 are measurable and aligned within the vented central space of the SMART container 401 without additional mechanical forces adding additional weight. As shown in FIG. 7, when in the disengaged configuration the multifunctional cap 501, medication bottle 601, and medication bottle may be removed from the central vented chamber of the SMART container, though they are still measurable until the horizontal tabs 509 of the multifunctional cap 501 are removed from atop of the strain gauges 410.

As shown in FIGS. 4a-c, in further preferred embodiments, the SMART container 401 further includes a user display interface 403 with relayed audible and/or visual interactions with the user occurring when the display screen 403 is touched in certain areas. Other preferred embodiments include voice activation to control interactions with the user. As detailed earlier, one or more LEDs 404 may be configured to emit light in order to provide health literacy, improve compliance, and for behavioral modification of users of the medication EID, including but not limited to use of language preferences and plain language terms the user may understand regarding the medication or substance prescribed and/or being taken, and in accordance with the taxonomy classification system.

The power and electrical systems 107, as shown in FIG. 1, includes but is not limited to its circuitry and its processor, which are integrative with the user interface's 103 audible and/or visual display 403 and LEDs 404, which are programmed with instructions to: receive 104 and store 108 input from the user interface 103; and display information 403 on the user interface 103. The circuitry is programmed with logic and/or instructions for performing the functions as recited herein.

Referring to FIGS. 1 to 3, the taxonomy classification system 105, compliance and behavioral modification systems 106, and power and electrical systems 107 are not particularly limited but preferably integrative and populated from diverse data points consisting of: information about medication present within the medication bottle 601 (e.g. type, amount, weight, storage conditions, side effects, body effects, alerts etc.), information about user demographics, information about a user's medical condition(s), and information regarding medication compliance, persistence, adherence, and supportive assistance needs.

In other preferred embodiments as shown in FIGS. 5a-c, the multifunctional cap 501 has a liquid medication dispensing portal 508 for fluid communication with a medication bottle, such as shown in FIG. 5a, wherein medication (liquid, solids, gas) weight may be determined through circuitry in communication with an algorithmic control logic system, such as system 108, shown in FIG. 1. In some embodiments, the multifunctional cap 501 connects with a medication bottle by engaging a friction fit central core 511 and lateral protrusions extending from a button 510 housed within the inner chamber of the multifunctional cap 505, wherein the lateral projections of the multifunctional cap button engages and disengages from the inner ridge 602 of the medication bottle 601, when the multifunctional cap 501 is manipulated toward and away from the central compartment of the medication bottle 601, respectively. The friction fit central core 511 is a part of the base of the multifunctional cap 501, such that the inner chamber 505 of the multifunctional cap 501 extends from the base of the friction fit central core 511 toward the nonrotating portion of the multifunctional cap 501, wherein medications may exit through the sidewall 508 and/or top 503 of the multifunctional cap 501.

As seen in FIG. 5b, the multifunctional cap 501 preferably has a nonrotating central internal chamber 505 that is funnel shaped 507 with ridges and/or inward projections of varying types (i.e., circular, tubular, rectangular, thin, dense, elongated, shortened) of obstructors 506 extending from the walls into the inner chamber to impede medications, like pills or tablets, from rapidly exiting the multifunctional caps' nonrotating inner chamber 505 and the external rotating lid 503, when the medication bottle 601 is rotated and/or shaken to allow medication 603 to exit through the side 508 and/or top 503 of the multifunctional cap. Specifically, the internal pill obstructors 506, which protrude into the inner chamber of the multifunctional cap disengage medications from one another, such that the solid medications, such as pills, capsules, and tablets, and liquids flow out of the side 508 and/or top 503 of the multifunctional cap one at a time and/or at slower rates. One embodiment has a mechanism wherein the rotating external lid 502 is adjusted to support medication exiting the nonrotating internal chamber 505 of the multifunctional cap 501 to allow for pills of differing sizes to exit. This additional feature may be useful for persons who use routine identical pill sizes and desire greater accuracy with 1 pill exiting the multifunctional cap.

In other preferred embodiments, an additive, such as a food-safe silicone, is contained within the walls of the funnel-shaped 507 inner chamber 505 of the multifunctional cap 501 for use as a medication desiccant and/or dehumidifier for medication preservation. In some embodiments the multifunctional cap includes safety and locking features for child safety.

In yet additional preferred embodiments, the enclosure and/or programmed circuity further comprises: an accelerometer 410 as show in FIG. 4a and/or thermometer for assessing movement and/or temperature of medication bottle and medication storage conditions, to among other things, assess the impact of temperature on medication quality and quality outcomes, as well as to assess, detect, diagnose, and/or intervene when changes in users' ease of use, difficulty of use, or unusual user environmental interactions occur with use of the medication EID 102, which may be undetected by medication EID users 111, healthcare providers 304, and approved social network members 306 without accelerometer use 409; and (b) strain gauges 410 for assessing proper dispensing compliance, calculating medication bottle contents 603, and determining if technical support is preemptively needed to improve the quality of medication EID 102 and remote sensing capabilities of the power and electrical systems 107.

Reference throughout the specification to “one embodiment,” “another embodiment,” “an embodiment,” “some embodiments,” and so forth, means that a particular element (e.g., feature, structure, property, and/or characteristic) described in connection with the embodiment is included in at least one embodiment described herein, and may or may not be present in other embodiments. In addition, it is to be understood that the described element(s) may be combined in any suitable manner in the various embodiments.

1. A medication environmental interactive device (EID), the EID comprising: a container having a container space for receiving a medication bottle; a multifunctional cap for attaching to the medication bottle; a weight sensor; a power supply; and programmed circuitry,
wherein:
the container has an interaction surface configured to receive and suspend the multi-functional cap and attached medication bottle within the container space;
the weight sensor is disposed between the interaction surface and the multi-functional cap, and is configured to measure the weight of the multifunctional cap, the medication bottle, and contents of the medication bottle; and
the programmed circuitry is programmed with instructions to receive and store information from the weight sensor.

2. The EID of claim 1, wherein the weight sensor comprises one or more strain gauges, wherein the one or more strain gauges are preferably formed from a metal alloy and/or carbons, including but not limited to graphene nanosheets synthesized using a water-assisted liquid phase exfoliation (GNS-LPE).

3. The EID of claim 2, wherein the multifunctional cap and the container are configured to reversibly engage between an engaged configuration and a disengaged configuration, wherein when in the engaged configuration the medication bottle attached to the multifunctional cap is enclosed and suspended within the container space and when in the disengaged configuration the multifunctional cap and prescription medication bottle can be removed from the container space.

4. The EID of claim 3, wherein the interaction surface comprises one or more u-shaped interaction surfaces (e.g. hollow strain gauge contact surfaces) and the multifunctional cap comprises one or more horizontal tabs, wherein when in the engaged configuration the one or more strain gauges are disposed between the one or more u-shaped contact surfaces of the container and the one or more horizontal tabs of the multifunctional cap.

5. The EID of claim 4, wherein the EID and/or multifunctional cap comprises a safety lock to prevent removal of the multifunctional cap from the container and/or medication bottle.

6. The EID of claim 5, wherein the safety lock comprises one or more rotatable top covers attached to EID and configured to rotate to lock the one or more tabs of the multifunctional cap in the one or more u-shaped interaction surfaces (hollow strain gauge contact surfaces).

7. The EID of claim 1, wherein the multifunctional cap comprises a medication dispensing portal and a desiccant, wherein the medication dispensing portal and desiccant are in fluid communication with the contents of the attached medication bottle.

8. The EID of claim 1, wherein the inner chamber of the multifunctional cap is funnel-shaped with ridges and/or inward facing projections/obstructors of varying types (i.e., circular, tubular, rectangular, thin, dense, elongated, shortened) to disengage medications from one another and/or reduce the number and/or volume of medications exiting the side and/or top of the multifunctional cap to reduce medication spills and improve accuracy of one pill exiting the multifunctional cap.

9. The EID of claim 1, wherein the medication bottle comprises a circumferential ridge and the multifunctional cap comprises horizontal projected tabs, wherein the horizontal tabs interact with the circumferential ridge of the medication bottle to secure the multifunctional cap to the medication bottle in a friction fit and/or snap fit arrangement.

10. The EID of claim 1, wherein the programmed circuitry further comprises:
a user interface comprising an audible and/or visual display;
wherein the programmed circuitry is programmed with instructions to: receive and store information received from the user interface and display information on the display;
wherein the information is selected from the group consisting of: information about medication present within the medication bottle, information about a user, information about a medical condition of a user, and information regarding medication disposed with the bottle.

11. The EID of claim 10, further comprising an accelerometer, a bar code scanner (e.g. linear, laser, 2D area bar code scanner), and a wireless transmitter, wherein the programmed circuitry is programmed with instructions to: receive and store information from the accelerometer, the bar code scanner, and wireless transmitter; and receive and send data to/from a remote server.

12. The EID of claim 10, wherein the user interface further comprises a LED, wherein the programmed circuitry is programmed with instructions to emit colored light based on medication contained within the bottle and/or user preferred information.

13. The EID of claim 10, wherein the programmed circuitry is programmed with instructions to auto-populate the user interface in an algorithmic derived manner and/or instruct a user through prompting for information/interactions regarding medical condition, patient information, and/or prescription medication administration and/or for providing information to the user regarding these topics.

14. A medication management system (MMS) comprising a medication EID, provisioned-network communication networks, and cloud-based servers, which may be used singularly and/or collectively facilitate use of a health-related and medication-related platform by incorporating a health literacy taxonomy classification system (HLTCS), wherein end-users are provided health-related and pharmacy-related information in their preferred language and plain language health literacy terms.

15. The MMS of claim 14, wherein the HLTCS is used in promoting health literacy integrated medication adherence monitoring/dosing reminder systems, which use unobtrusive behavior modification interventions.

16. The MMS of claim 14, wherein the HLTCS is used in conjunction with machine learning, artificial intelligence, neural networks, national language processing, and other applicable advanced data science methodologies to detect, assess, and eliminate harmful bias.

17. The MMS of claim 14, wherein the HLTCS is used to promote social engagement of care through use of preferred health literacy language of medical conditions and pharmacy-related information to assist providers and supportive care teams (family, providers, pharmacists, government, etc.) in management of medication for medication EID users.

18. The EID of claim 13 and the MMS of claim 14, wherein the programmed circuitry is programmed with integrative (a) taxonomy classification systems, (b) compliance and behavioral modification systems, and (c) power and electronics systems auto-populate a user interface by integrating medical conditions, prescribed medicines, and preferred health literacy language preferences.
